(19) United States
(12) Patent Application Publication
(10) Pub. No.: US 2024/0054316 A1
(43) Pub. Date: Feb. 15, 2024

(54) BIOCHEMICAL REACTION LOGIC GATE BASED ON ENZYMATIC REACTION

(71) Applicant: South China University of Technology, Guangzhou (CN)

(72) Inventors: Han HUANG, Guangzhou, Guangdong (CN); Chengzhi MA, Guangzhou, Guangdong (CN); Tongli HE, Guangzhou, Guangdong (CN); Xinglong XIAO, Guangzhou, Guangdong (CN)

(73) Assignee: South China University of Technology, Guangzhou (CN)

(21) Appl. No.: 18/259,158

(22) PCT Filed: Feb. 7, 2021
(86) PCT No.: PCT/CN2021/075708

§ 371 Date: Jun. 23, 2023

(30) Foreign Application Priority Data
Dec. 24, 2020 (CN) 202011550421.3

(51) Int. Cl. G06N 3/00

(52) U.S. Cl. CPC G06N 3/002

(57) ABSTRACT

A biochemical reaction logic gate based on an enzymatic reaction, wherein input and output signals of the biochemical reaction logic gate are the concentrations or activities of substances and an enzyme in a reaction system. The logic implementation and the cascade of the logic gate are based on a plurality of enzymatic reactions. When the concentrations or activities of the substances which represent the input signal change, the concentrations of a substrate and a product of the enzymatic reaction change, and the concentrations of the other substances change accordingly. The biochemical reaction logic gate has the characteristics of reusability, cascading, a low delay, and a low power consumption, is a basic component for forming computer logic, and can be used for constructing a biochemical reaction central processing unit and a biochemical reaction computer.

### CROSS-REFERENCE TO RELATED APPLICATIONS

The present application is a national phase entry under 35 USC § 371 of International Application PCT/CN2021/075708, filed Feb. 7, 2021, which claims the benefit of and priority to Chinese Patent Application No. 2020115504213, filed Dec. 24, 2020, the entire disclosures of which are incorporated herein by reference.

### TECHNICAL FIELD

The present disclosure relates to the technical field of bio-computers, in particular to a biochemical reaction logic gate based on an enzymatic reaction.

### BACKGROUND

At present, it is a subject with great potential to build a bio-computer with low power consumption and high performance by imitating the brain of an organism, especially the brain of a human. A basic unit logic gate required for building a bio-computer is implemented by means of a substance with certain biochemical activity. At present, the proposed biochemical reaction logic gates are generally based on DNA substitution reactions, living cell transcription and translation systems, and protein dimer reactions based on hydrogen bonding. Biochemical reaction logic gates based on DNA substitution reactions and on living cell transcription and translation systems are both based on the principle of base pair pairing, so the reaction rate is slow, which leads to the delay of the above biochemical reaction logic gates generally ranging from hours to days. A protein dimer reaction based on hydrogen bond pairing is essentially a reaction in which hydrogen bonds are broken and recombined due to the different strengths of different hydrogen bond pairing. Therefore, once all hydrogen bonds have been completely recombined to make the system reach a stable state with the lowest energy, the biochemical reaction logic gate based on this principle can no longer be used, and it is not reusable; in addition, due to the recombination of hydrogen bonds, the substance originally used as the input signal will be modified, resulting in a damaged input signal. In summary, the existing biochemical logic gates have problems such as poor cascading property, long gate delay, non-reusability, and damaged signal input.

### SUMMARY

An object of the present disclosure is to provide a biochemical reaction logic gate based on an enzymatic reaction, to solve the defects of the existing biochemical logic gates, such as large delay and non-cascading. In the biochemical reaction logic gate of the present disclosure, a concentration of a substance and an activity of an enzyme are used as input and output, and a change in the activity of the enzyme drives the concentration and activity of other substances and enzymes to change, thus realizing logical calculation and gate cascading. Since an enzymatic reaction can be quickly completed under enzyme catalysis, the logic gate provided by the present disclosure can reach a delay on the level of milliseconds to picoseconds, which can well solve the defect of high delay, while the process of DNA transcription and translation on which other biochemical logic gate technologies are based is slow, the delay of the existing biochemical logic gates ranges from hours to days. In addition, compared with other biochemical logic gates based on proteins, the enzymatic reaction provided by the present disclosure can be recycled and cascaded in multiple stages due to its reversibility and cascading property.

The object of the present disclosure can be achieved by using the following technical solution.

A biochemical reaction logic gate based on an enzymatic reaction is provided. The biochemical reaction logic gate is based on a plurality of enzymatic reactions, concentrations or activities of a substance and an enzyme are used as input and output signals of the logic gate, and a change in the activity of the enzyme causes a change in concentrations of a corresponding substrate and a product, thus realizing a logic and a gate cascading of the logic gate.

In the biochemical reaction logic gate, the concentration or activity of the substance or enzyme, when used as an output signal, is affected by the activity of other enzymes. The activity of the enzyme may be used as an input signal, and when the activity of the enzyme changes, the concentrations of the substrate and product of the corresponding enzymatic reaction, as an output signal, change. As an input signal of the biochemical reaction logic gate, the concentration of the enzyme may be affected and controlled in real time by an electrical signal. As an output signal of the biochemical reaction logic gate, the concentration of the substance may be detected and converted into an electrical signal in real time.

Preferably, the substrate or product of the enzymatic reaction may also be used as an enzyme to catalyze other reactions. On the one hand, the substrate and product are affected by the activity of the enzyme and act as an output signal; on the other hand, the substrate and product can catalyze other reactions, thus affecting other reactions and acting as an input signal of the next gate, so the biochemical reaction logic gate has cascading property.

Preferably, each enzymatic reaction in the biochemical reaction logic gate has another reverse reaction corresponding thereto, which satisfies the requirement of consuming the product of the enzymatic reaction and generating the substrate of the enzymatic reaction. Therefore, as the activity of the enzyme increases, the rate of the enzymatic reaction increases, the concentration of the substrate decreases, and the concentration of the product increases; whereas, as the activity of the enzyme decreases, the rate of the enzymatic reaction decreases, and the reverse reaction goes on simultaneously, so the concentration of the substrate increases and the concentration of product decreases. Therefore, the gate logic can be implemented by changing the activity of the enzyme to drive the change of the concentrations of the substrate and product. Moreover, since the reverse reaction can reuse the product of the enzymatic reaction, the biochemical reaction logic gate provided by the present disclosure is reusable.

Preferably, the concentration or activity of a substance may be directly or indirectly affected simultaneously by the concentration or activity of a plurality of enzymes, so that the concentration or activity of this substance is controlled simultaneously by the concentration or activity of a plurality of substances, thereby realizing a biochemical reaction logic gate. The concentration or activity of a controlled substance is the output signal of the biochemical reaction logic gate, and the concentrations or activities of a plurality of substances that directly or indirectly control the output signal are a plurality of input signals of the biochemical reaction logic gate, and the plurality of input signals simultaneously affect the input signals, thereby realizing the logical operation of the biochemical reaction logic gate. In the biochemical reaction logic gate, the concentration or activity of an enzyme is used as an input signal to control the output signal, and the concentration or activity of the enzyme is not affected during the enzymatic reaction, so the biochemical reaction logic gate provided by the present disclosure has the characteristics of feedforward transmission of the input signal and keeping the input signal undamaged. The biochemical reaction logic gate provided by the present disclosure has the technical advantages of reusability, cascading, signal feedforward transmission and no damage to input, meets all requirements for constructing combinational logic and sequential logic circuits like traditional semiconductor logic gates, and can be used for constructing biochemical reaction central processors and biochemical reaction computers.

Compared with the prior art, the present disclosure has the following advantages and effects.
(1) The biochemical reaction logic gate based on an enzymatic reaction as provided by the present disclosure can be conveniently cascaded to form a complex logic because both the input and output of the biochemical reaction logic gate are the concentrations of an enzyme or a substrate.
(2) Since the reaction rate of the enzymatic reaction in the biochemical reaction logic gate based on an enzymatic reaction as provided by the present disclosure is fast, the delay of the gate can be as low as milliseconds to picoseconds, thereby achieving fast operation.
(3) Since the mass of the enzyme in the biochemical reaction logic gate based on an enzymatic reaction as provided by the present disclosure has no loss before and after the reaction and the substances involved in the reaction can also be reused, the logic gate can also be reused and realize signal feedforward transmission, which overcomes the shortcomings of the existing biochemical logic gates and meets the requirements of building biochemical computers.

### BRIEF DESCRIPTION OF DRAWINGS

FIG. 1 is a schematic diagram of a biochemical reaction logic gate—OR gate disclosed in an example of the present disclosure; and

FIG. 2 is a schematic diagram of a biochemical reaction logic gate—AND gate disclosed in an example of the present disclosure.

### DETAILED DESCRIPTION

In order to make the object, technical solution and advantages of the examples of the present disclosure more clear, the technical solution in the examples of the present disclosure will be described clearly and completely below in conjunction with the attached drawings of the examples of the present disclosure. Obviously, the described examples are some, not all of the examples of the present disclosure. Based on the examples in the present disclosure, all other examples obtained by those of ordinary skill in the art without involving any inventive effort fall within the scope of protection of the present disclosure.

### Example 1

This example implemented a biochemical reaction logic gate—OR gate. The implementation of the OR gate requires three groups of phosphorylation reactions, wherein E1 is a phosphorylase that catalyzes a first reaction, S1 is a substrate of the first reaction, and S1′ is a product of the first reaction; E2 is a phosphorylase that catalyzes a second reaction, S2 is a substrate of the second reaction, and S2′ is a product of the second reaction; and E3 is a phosphorylase that catalyzes a third reaction, S3 is a substrate of the third reaction, S3′ is a product of the third reaction, and PP3 is a dephosphorylase that catalyzes the reverse reaction of the third reaction. Phosphorylase E1 phosphorylates and deactivates substrate S1, phosphorylase E2 phosphorylates and deactivates substrate S2, and phosphorylase E3 phosphorylates and deactivatessubstrate S3. However, dephosphorylase PP3 may remove the phosphate group from the substrate inactivated by E3 phosphorylation and restore the activity of the substrate.

These three groups of reactions were used for kinase editing to construct an enzymatic reaction network and construct an OR gate. S1 was covalently bound to E3, and the binding product thereof was denoted as S1-E3; S2 was covalently bound to E3, and the binding product thereof was denoted as S2-E3. When S1-E3, S2-E3, S3 and PP3 were mixed in the same solution, the content of the product S3-P was controlled by the contents of S1-E3 and S2-E3, and the content of PP3 remained constant during operation. The content of S3-P was the output of the gate, S1-E3 and S2-E3 were the input of the gate, and the OR or AND logic of the gate depended on the content of PP3.

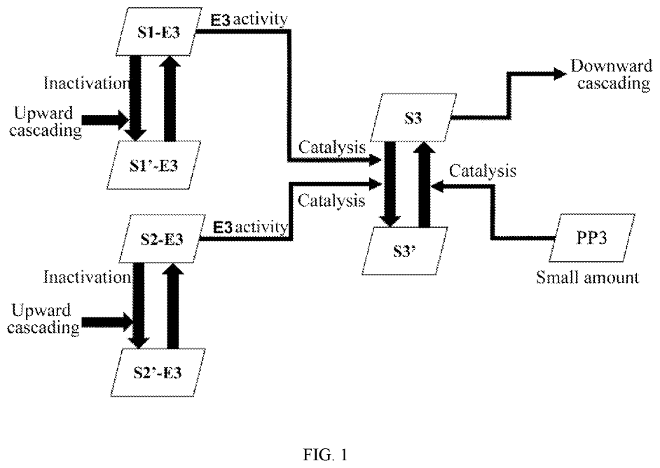

As shown in FIG. 1, when the content of PP3 is low, the degree of dephosphorylation is low, and the decomposition rate of S3′ is low, and a large amount of products can be generated under catalysis by only a small amount of kinase, so when one of the input S1-E3 or S2-E3 has a high content, the content of the product may be high (output logic 1), and only both inputs S1-E3 and S2-E3 are low, the content of the product is low (output logic 0), thus realizing the logic of OR gate.

### Example 2

This example implemented a biochemical reaction logic gate—AND gate. The implementation of the AND gate requires three groups of phosphorylation reactions, wherein E1 is a phosphorylase that catalyzes a first reaction, S1 is a substrate of the first reaction, and S1′ is a product of the first reaction; E2 is a phosphorylase that catalyzes a second reaction, S2 is a substrate of the second reaction, and S2′ is a product of the second reaction; and E3 is a phosphorylase that catalyzes a third reaction, S3 is a substrate of the third reaction, S3′ is a product of the third reaction, and PP3 is a dephosphorylase that catalyzes the reverse reaction of the third reaction. Phosphorylase E1 phosphorylates and deactivates substrate S1, phosphorylase E2 phosphorylates and deactivates substrate S2, and phosphorylase E3 phosphorylates and deactivates substrate S3. However, dephosphorylase PP3 may remove the phosphate group from the substrate inactivated by E3 phosphorylation and restores the activity of the substrate.

The three groups of reactions were used for kinase editing to construct an enzymatic reaction network and construct an AND gate. S1 was covalently bound to E3 to obtain S1-E3; S2 was covalently bound to E3 to obtain S2-E3. When S1-E3, S2-E3, S3 and PP3 were mixed in the same solution, the content of the product S3′ was controlled by the contents of S1-E3 and S2-E3, and the content of PP3 remained constant during operation. The content of S3′ was the output of the gate, S1-E3 and S2-E3 were the inputs of the gate, and the OR or AND logic of the gate depended on the content of PP3.

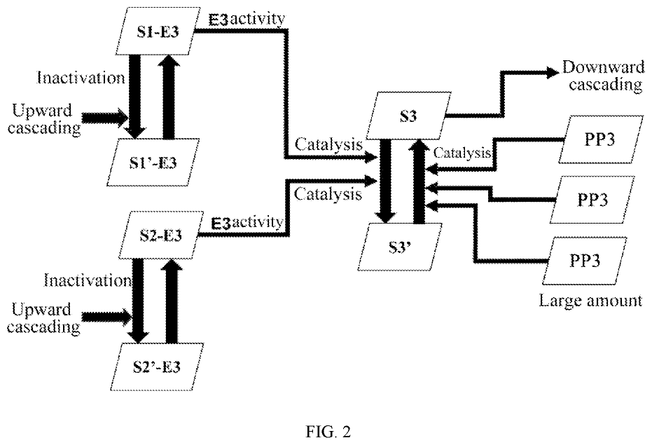

As shown in FIG. 2, when the content of PP3 is high, the degree of dephosphorylation is high, and the decomposition rate of S3′ is high, and a large amount of kinase was needed for catalysis to produce a large number of products, so only when the inputs of the contents of the two substances S1-E3 and S2-E3 are both high, the product content can become high (output logic 1), and as long as one of the inputs is low, the product content is low (output logic 0), thus realizing the logic of AND gate.

The above-mentioned examples are preferred embodiments of the present disclosure. However, the embodiments of the present disclosure are not limited by the above-mentioned examples, and any other changes, modifications, substitutions, combinations and simplifications made without departing from the spirit and principle of the present disclosure shall all be equivalent substitutions and are all included in the scope of protection of the present disclosure.

1. A biochemical reaction logic gate based on an enzymatic reaction, wherein the biochemical reaction logic gate is based on a plurality of enzymatic reactions carried out in the same solution; an input signal of the biochemical reaction logic gate is concentrations or activities of a substance and an enzyme; an output signal of the biochemical reaction logic gate is the concentrations or activities of the substance and the enzyme; and a logic and a cascade of the biochemical reaction logic gate are implemented by a change in concentrations of a corresponding substrate and a product caused by a change in the activity of the enzyme.

2. The biochemical reaction logic gate based on the enzymatic reaction according to claim 1, wherein the change in the concentration or activity of the substance is affected by the concentration or activity of one or more enzymes.

3. The biochemical reaction logic gate based on the enzymatic reaction according to claim 1, wherein the concentration or activity of the substance for the input signal of the biochemical reaction logic gate is changed and controlled in real time via an electrical signal.

4. The biochemical reaction logic gate based on the enzymatic reaction according to claim 1, wherein the concentration or activity of the substance for the output signal of the biochemical reaction logic gate is detected and converted into an electrical signal in real time.

5. The biochemical reaction logic gate based on the enzymatic reaction according to claim 1, wherein another reaction is present in a solution, in which the enzymatic reaction is conducted, and consumes a product of the enzymatic reaction and generate a substrate of the enzymatic reaction.

6. The biochemical reaction logic gate based on the enzymatic reaction according to claim 1, wherein when the activity of the enzyme changes, the concentrations of the substrate and product of the enzymatic reaction also change accordingly.

7. The biochemical reaction logic gate based on the enzymatic reaction according to claim 1, wherein the substrate and product of the enzymatic reaction are used as enzymes to catalyze another enzymatic reaction.
